(19) United States
(12) Patent Application Publication
(10) Pub. No.: US 2025/0099155 A1
(43) Pub. Date: Mar. 27, 2025

(54) ELECTROSURGICAL SYSTEM WITH ADAPTIVE NON-THERMAL PLASMA CONTROL

(71) Applicant: US Patent Innovations, LLC, Takoma Park, MD (US)

(72) Inventors: Jerome Canady, Lakeland, FL (US); Changhan Jun, Beltsville, MD (US); Taisen Zhuang, Rockville, MD (US); Buddika Sumanasena, Silver Spring, MD (US); Yang Wu, Takoma Park, MD (US); Feng Yan, Fairfax, VA (US); Priscilla Rubio, Takoma Park, MD (US)

(21) Appl. No.: 18/832,311

(22) PCT Filed: Feb. 3, 2023
(86) PCT No.: PCT/US2023/012342

§ 371 Date: Jul. 23, 2024

Related U.S. Application Data
(60) Provisional application No. 63/306,392, filed on Feb. 3, 2022.

(51) Int. Cl. A61B 18/04; A61B 18/00

(52) U.S. Cl. CPC A61B 18/042; A61B 2018/00178; A61B 2018/00648; A61B 2018/0072; A61B 2018/00726; A61B 2018/00827; A61B 2018/00892; A61B 2018/0091

(57) ABSTRACT

A system for adaptive control of non-thermal plasma treatment on a patient. The system has a electrosurgical generator, having a power module, a memory, a first current sensor configured to sense an electrical current on the active electrode, a second current sensor configured to sense a current on the return electrode, a voltage sensor configured to sense a voltage difference between the active electrode and the return electrode, and a feedback control module, wherein the control module is configured to automatically adjusts a power output of the electrosurgical generator based on outputs of the first current sensor, the second current sensor and the voltage sensor to maintain an electrical current on the active electrode within a range to provide non-thermal plasma.

### CROSS-REFERENCE TO RELATED APPLICATIONS

The present application claims the benefit of the filing date of U.S. Provisional Patent Application Ser. No. 63/306,392 filed by the present inventors on Feb. 3, 2022.

The aforementioned provisional patent application is hereby incorporated by reference in its entirety.

### STATEMENT REGARDING FEDERALLY SPONSORED RESEARCH OR DEVELOPMENT

None.

### BACKGROUND OF THE INVENTION

### Field Of The Invention

The present invention relates to electrosurgical systems and methods, and more particularly, to a surgical plasma accessory with distance sensing or adaptive non-thermal plasma control to produce non-thermal plasma.

### Brief Description Of The Related Art

The standard means for controlling traumatic and surgical blood loss are electrosurgical generators and lasers which respectively direct high-frequency electrical currents or light energy to localize heat in bleeding vessels so as to coagulate the overlying blood and vessel walls. Hemostasis and tissue destruction are of critical importance when removing abnormal tissue during surgery and therapeutic endoscopy. For monopolar electrosurgery electrical energy originates from an electrosurgical generator and is applied to target tissue via an active electrode that typically has a small cross-sectional surface-area to concentrate electrical energy at the surgical site. An inactive return electrode or patient plate that is large relative to the active electrode contacts the patient at a location remote from the surgical site to complete and electrical circuit through the tissue. For bipolar electrosurgery, a pair of active electrodes are used and electrical energy flows directly through the tissue between the two active electrodes.

U.S. Pat. No. 4,429,694 to McGreevy disclosed a variety of different electrosurgical effects that can be achieved depending primarily on the characteristics of the electrical energy delivered from the electrosurgical generator.

Another method of monopolar electrosurgery via argon plasma technology was described by Morrison U.S. Pat. No. 4,040,426 in 1977 and McGreevy U.S. Pat. No. 4,781,175. This method, referred to as argon plasma coagulation (APC) or argon beam coagulation is a non-contact monopolar thermoablative method of electrocoagulation that has been widely used in surgery for the last twenty years. In general, APC involves supplying an ionizable gas such as argon past the active electrode to target tissue and conducting electrical energy to the target tissue in ionized pathways as non-arcing diffuse current. In U.S. Patent Application Publication No. 2013/0296848, Canady et al. described electrosurgical systems and methods using argon plasma during cutting modes of operation. In such gas-assisted electrosurgical systems a probe typically is held within 1 cm of the target tissue to maintain plasma discharge to the tissue to produce the desired coagulating effect.

U.S. Pat. No. 10,052,146 disclosed an electrosurgical method and device for simultaneously cutting and coagulating tissue with an electrosurgical device having an electrode and a channel wherein the channel has a port near a proximal end of the electrode, wherein the method comprises the steps of causing an inert gas to flow through the channel and exit the port, applying high-frequency energy to the electrode while the inert gas flows through the channel, wherein the high-frequency energy applied to the electrode continuously plasmatizes inert gas exiting the port, initiating an electrical discharge from the electrode through the continuously plasmatized inert gas to the tissue, cutting tissue with the electrode, maintaining the electrical discharge from the electrode through the plasmatized inert gas while cutting tissue with the electrode to cause coagulation of the tissue simultaneously with the cutting.

Another electrosurgical system is disclosed in U.S. Pat. No. 10,064,675, entitled “Multi-mode Electrosurgical Apparatus.”

Plasma is an ionized gas that is typically generated in high-temperature laboratory conditions. Recent progress in atmospheric plasmas has led to the creation of cold or non-thermal plasmas with ion temperature close to room temperature, i.e., less than about 37° C. Earlier studies demonstrated the non-aggressive nature of the cold plasma. After it was shown, albeit indirectly, that plasma can interact with organic materials without causing thermal/electric damage to the cell surface, several biological applications were examined. Non-thermal or cold plasmas have an increasing role to play in biomedical applications. The potential use in biomedical applications has driven the development of a variety of reliable and user-friendly plasma sources.

U.S. Pat. No. 10,213,614 disclosed a dielectric barrier discharge device (DBD) that generates cold plasma to treat cancerous tumors. The device has a gas supply tube with a delivery end. The gas supply tube is configured to carry a gas to the delivery end. A syringe is provided having an opening. The syringe is connected to the supply tube and configured to carry the gas to the opening. A first electrode is positioned inside the syringe, and a second electrode is positioned adjacent to the opening. The first and second electrodes excite the gas to enter a cold plasma state prior to being discharged from the opening of the syringe. An endoscopic tube can be used instead of the syringe. An exhaust tube can be provided to remove gas introduced into the body cavity by the cold plasma jet.

U.S. Pat. No. 9,999,462 disclosed a system that does conversion from regular thermal plasma produced by an electrosurgical unit (ESU) to non-thermal cold plasma, which is thermally harmless to healthy tissue. The system is comprised of conversion unit and cold plasma probe. The output signal from the ESU connects to a conversion unit. The conversion converts a high-frequency electro-surgical signal from the ESU to a low-frequency signal and sends it to the output connector along with helium flow. The cold plasma probe is connected directly to the conversion unit output, and cold plasma is produced at the end of the cold plasma probe.

Additional developments in gas-assisted electrosurgical systems are disclosed in U.S. Pat. No. 11,020,541 entitled “Electrosurgical Gas Control Module,” and U.S. Pat. No. 11,253,310, entitled “Gas-enhanced Electrosurgical Generator.”

### SUMMARY OF THE INVENTION

In a preferred embodiment, the present invention is a system for performing non-thermal plasma treatment on a patient. The system has a electrosurgical generator for supplying radio frequency energy, a return electrode connected to the electrosurgical generator, a gas control module for controlling a supply of an inert gas, and a hand piece having a proximal end and a distal end. The hand piece has a housing having an inner cavity, tubing within the cavity, a fluid connector connecting the tubing to the gas control module, an active electrode within the cavity, an electrical connector connecting the active electrode to the electrosurgical generator, and a printed circuit board within the cavity and connected to the conductor. The electrosurgical generator has a power module, a memory, a first current sensor configured to sense an electrical current on the active electrode, a second current sensor configured to sense a current on the return electrode, a voltage sensor configured to sense a voltage difference between the active electrode and the return electrode, and a feedback control module, wherein the control module is configured to automatically adjusts a power output of the electrosurgical generator based on outputs of the first current sensor, the second current sensor and the voltage sensor to maintain an electrical current on the active electrode within a range to provide non-thermal plasma. During operation, an inert gas flows from a source of inert gas through the gas control module and through the tubing in the hand piece. Electrosurgical energy is provided by the electrosurgical generator to the active electrode, at least a portion of which is exposed to the inert gas that flows through the tubing in the hand piece. The electrosurgical energy is provided in a current range to plasmatize the inert gas that flows out of the hand piece to produce a non-thermal (<37° C.) plasma. The feedback control module monitors the current on the active electrode and the current on the return electrode as well as the voltage difference between the active and return electrodes and adjusts the power output of the electrosurgical generator to maintain the current on the active electrode within a range that produces non-thermal plasma. The active electrode may be a wire, or the active electrode and the tubing in the hand piece are integrated as a tubular conductor. The gas module may be integrated into the electrosurgical generator. The electrosurgical generator may include a flyback transformer and the feedback control module cause the flyback transformer to adjust the duty cycle of the electrical signal to maintain the current on the active electrode in the range to provide non-thermal plasma. The return electrode comprises a patient pad.

In another embodiment, the present invention is a method for producing non-thermal plasma for treatment on a patient. The method comprises the steps of generating an electrical current on an active electrode in an electrosurgical accessory with an electrosurgical generator, sensing the electrical current on the active electrode with a first current sensor in the electrosurgical generator, sensing an electrical current on a return electrode with a second current sensor in the electrosurgical generator, sensing a difference between a first voltage on of the active electrode and a second voltage on the return electrode with a voltage sensor, and automatically adjusting a power output of the electrosurgical generator based on outputs of the first current sensor, the second current sensor and the voltage sensor to maintain an electrical current on the active electrode within a range to provide non-thermal plasma.

Still other aspects, features, and advantages of the present invention are readily apparent from the following detailed description, simply by illustrating a preferable embodiments and implementations. The present invention is also capable of other and different embodiments and its several details can be modified in various obvious respects, all without departing from the spirit and scope of the present invention. Accordingly, the drawings and descriptions are to be regarded as illustrative in nature, and not as restrictive. Additional objects and advantages of the invention will be set forth in part in the description which follows and in part will be obvious from the description or may be learned by practice of the invention.

### BRIEF DESCRIPTION OF THE DRAWINGS

For a more complete understanding of the present invention and the advantages

thereof, reference is now made to the following description and the accompanying drawings, in which:

FIG. 1 is a perspective view of an electrosurgical accessory having a cold plasma scalpel.

FIG. 2 is a perspective view of a cold plasma scalpel and electrosurgical hand piece.

FIG. 3 is an assembly view of a cold plasma scalpel.

FIG. 4A is a schematic diagram of an exemplary gas-assisted electrosurgical system for use with the present invention.

FIG. 4B is a schematic diagram of an exemplary gas-assisted electrosurgical control system for use with the present invention.

FIG. 5 is an overview of an adaptive non-thermal plasma control system in accordance with a preferred embodiment of the present invention.

FIG. 6 is a circuit diagram of an adaptive cold plasma control circuit in accordance with a preferred embodiment of the present invention.

FIG. 7 is an illustration of a system having adaptive cold plasma control in accordance with a preferred embodiment of the present invention.

FIG. 8 is an exemplary hardware architecture in accordance with an alternative embodiment with a distance sensor on an electrosurgical hand piece.

FIG. 9 is a schematic diagram of a flexible printed circuit board in accordance with an alternative embodiment with a distance sensor on an electrosurgical hand piece.

FIG. 10 is a perspective view of a flexible printed circuit board in accordance with an alternative embodiment with a distance sensor on an electrosurgical hand piece.

FIG. 11 is a perspective view of a hand piece of an electrosurgical hand piece an alternative embodiment with a distance sensor on an electrosurgical hand piece.

FIG. 12 is a flow chart of a hand-held application in accordance with an alternative embodiment with a distance sensor on an electrosurgical hand piece.

FIG. 13 is a flow chart of a robotic application in accordance with an alternative embodiment with a distance sensor on an electrosurgical hand piece.

FIG. 14 is a flow chart of a real-time cold plasma dosage control application in accordance with an alternative embodiment with a distance sensor on an electrosurgical hand piece.

FIG. 15 is a flow chart of a cold plasma real-time treatment time control application in accordance with an alternative embodiment with a distance sensor on an electrosurgical hand piece.

FIG. 16 is an exemplary architecture of a system with 3D depth mapping using a robotic sensor probe.

### DETAILED DESCRIPTION OF THE PREFERRED EMBODIMENTS

Recent studies by the inventors have shown that a single device can deliver thermal plasma when held within 1 cm of target tissue and deliver non-thermal (cold) plasma at less than 37° C. at distances greater than 1 cm, e.g., between 1-2 cm from the target tissue. Maintaining a desired range of distance from the target tissue, however, can be difficult. A distance sensing system is one solution. Another solution, however, is an adaptive control system that dynamically adjusts the power output of the electrosurgical generator to ensure that the current being the tissue is low enough to deliver only non-thermal plasma with the plasma accessory held at a range of distances from the target tissue.

A preferred embodiment of a cold plasma system having an adaptive control system according to the present invention is described with reference to the figures. A handpiece such as is disclosed in U.S. Pat. No. 11,464,558, may be used. Such a handpiece includes a printed circuit board (PCB) that may include the circuitry to achieve the adaptive control, or separate circuitry may be used.

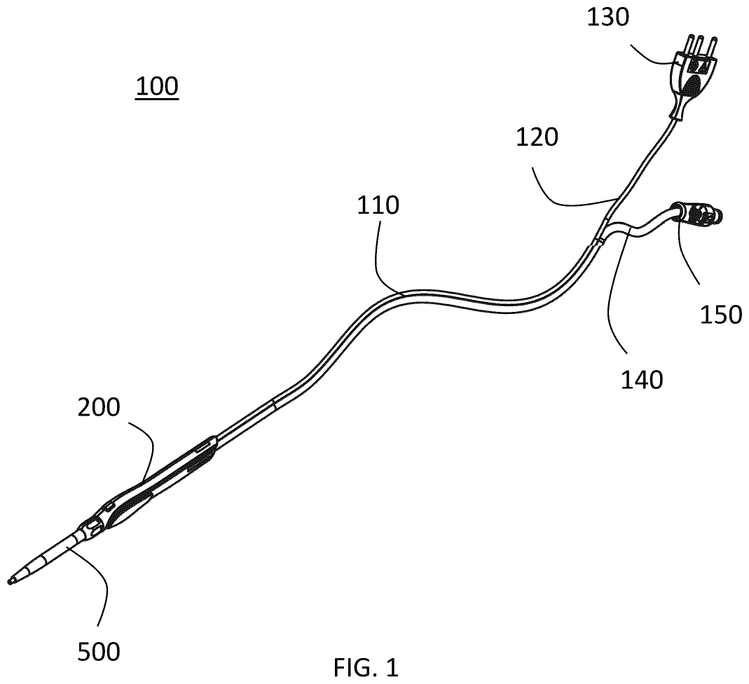

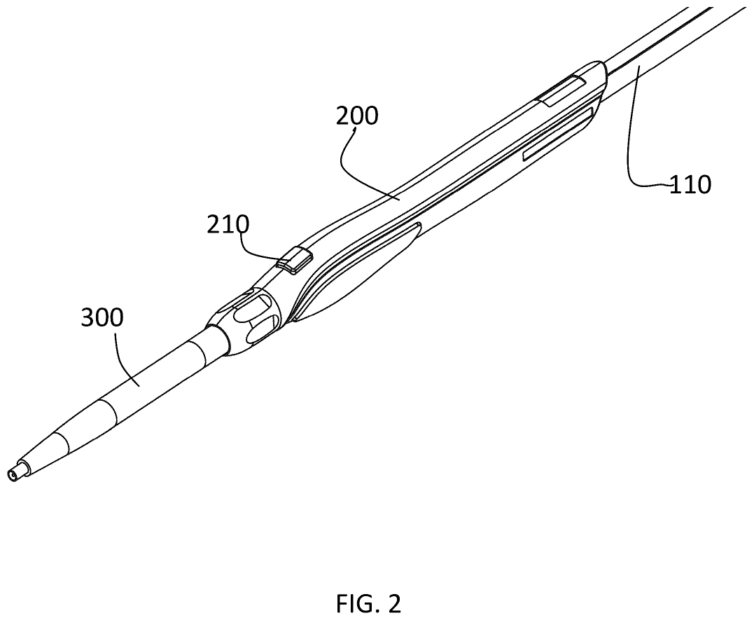

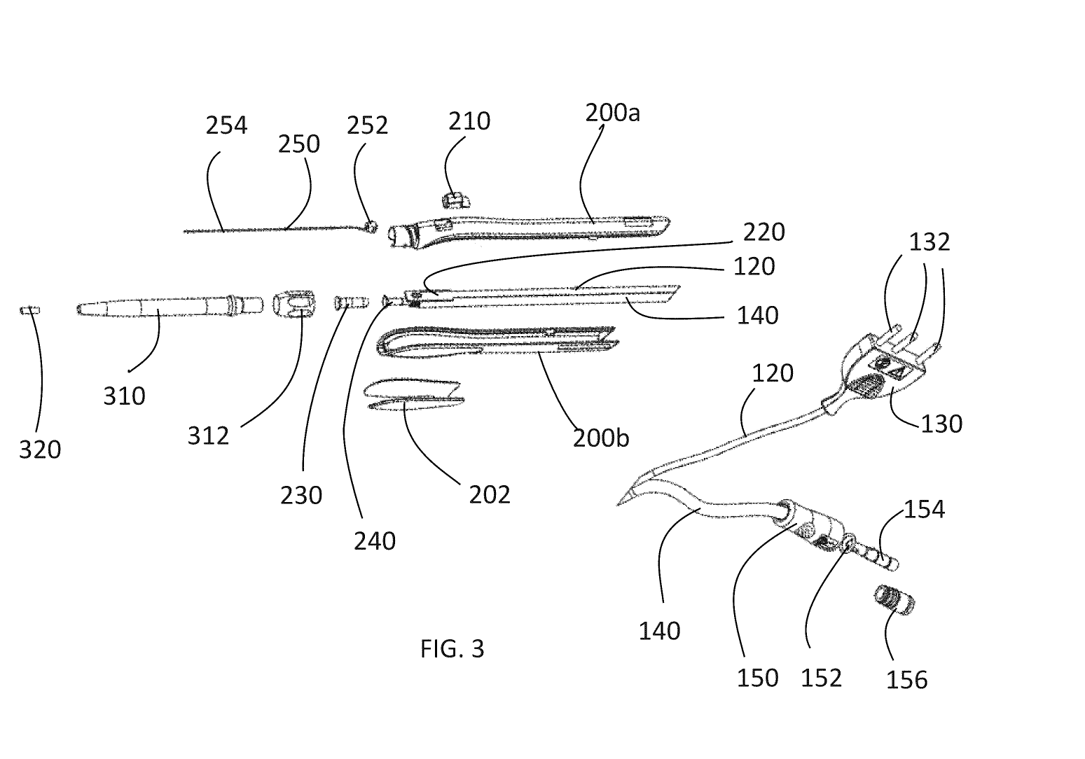

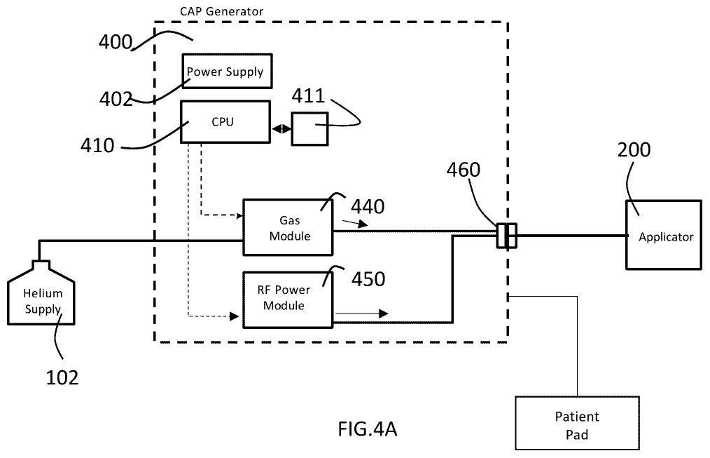

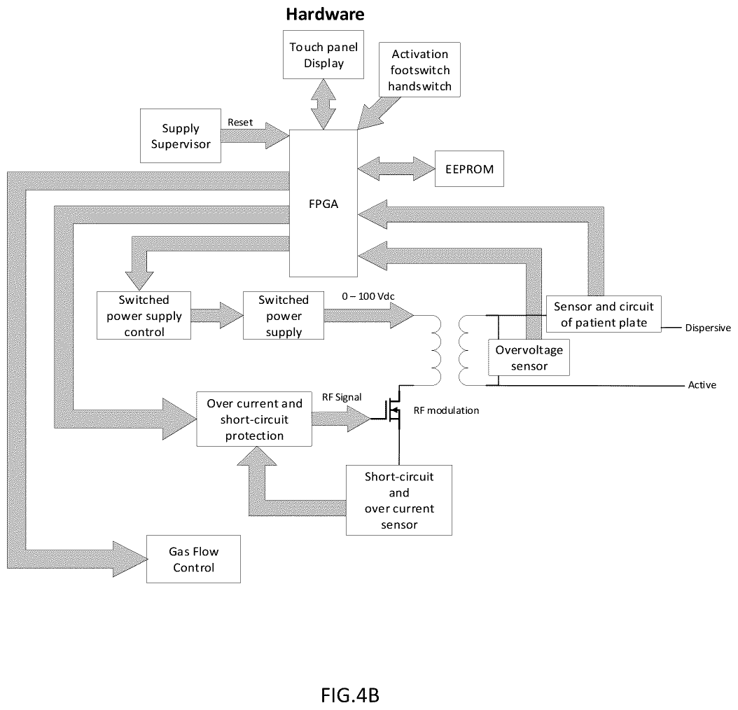

As shown in FIGS. 1-3, a hand piece assembly 200 has a top side piece 200a and a bottom side piece 200b. A control button 210 extends from the interior of the hand piece through an opening in the top side piece 200a. Within the hand piece is body connector funnel 230, PCB board 220, electrical wiring 120 and hose tubing (PVC medical grade) 140. The wiring 120 and hose tubing 140 are connected to one another to form a wire and tubing bundle 110. A grip over mold 202 extends over the bottom piece portion 200b. In other embodiments, a grip may be attached to the bottom piece 200b in other manners. A probe or scalpel assembly 300 is attached to the end of the hand piece. The scalpel assembly 300 has non-bendable telescoping tubing 310, a ceramic tip 320, a column nut or collet 312 and body connector tubing 240. The hose tubing 140 extends out of the proximal end of the hand piece to a body gas connector 150, which has an O- ring 152, gas connector core 154 and gas connector tip 156 for connecting to a source of gas (102 in FIG. 4). The printed circuit board 220 connects to electrical wiring 120 which leads to electrical connector 130 having electrical pins 132.

The collet 312, has a body having a plurality of depressions or dimples on its exterior for gripping the collet. The interior of the collet 312 has threads for engaging with threads on the hand piece 200.

The hand piece 200 has a housing having an upper portion 200a and a lower portion. The upper portion has a body having an opening for receiving a control button. Although only one opening is shown in this embodiment, other embodiments with additional openings for additional control buttons will be apparent to those of skill in the art. The upper portion 200a has a ridge structure along its sides and tabs for mating with the bottom portion 200b. The upper portion 200a has a neck having threads for mating with the threads on the collet 312. The neck additionally may have a self-alignment feature. On the interior of the upper portion 200a adjacent the hole is a pair of support elements for supporting PCB board 220 and control button 210 for controlling gas flow and/or electricity. Additional support elements support the PCB board. A foot pedal or pedals may be used in addition or instead of button 210 to control gas flow and/or electricity. The bottom portion 200b of the hand piece 200 has a body a pair of grip portions or a grip over mold 202, grooves for engaging with tabs in the upper portion 200a, and a ridge structure for engaging with the ridge structure in the upper portion 200a of the hand piece 200, and a slot for engaging with tab in the top piece.

The scalpel assembly 300 has a housing or body 310 having a channel within it, a ceramic tip 320, a column nut or collet 312. The housing 310 has a tapered portion near its distal end, an outer shoulder and a pair of inner shoulders. The housing or body 310 may be telescoping and may be comprised of telescoping tubing. The housing or body 310 has a proximal end that connects to the hand piece 200 by means of collet 312 and a distal end extending away from the hand piece 200. Within the proximal end of the housing or body 310 is a lip, flange or other support member for receiving an electrode connector. The telescoping tubing 310 may be of any length from a few millimeters to tens of centimeters or longer.

An electrode 250 is inserted into the distal end of the tubing, housing or body 510. The electrode has a connector and a wire or elongated portion. The connector and wire may be formed from the same or different materials. For example, the connector may be nickel-plated brass and the wire tungsten. The connector is at the proximal end of the electrode and has a connector body 252 having a beveled or rounded distal end and a proximal end. The connector may generally be cylindrical in shape but may have a flat portion for alignment of the electrode in the housing 510. The body 252 has a channel extending through it and a ridge, shoulder or flange. The wire 254 of the electrode is connected to the distal end of the connector adjacent the channel and extends from the distal end of the connector. The wire 254 may have a bent portion to position the end of the distal end of the wire 254 in the center of the channel in the body 310.

When the attachment 300 is fully assembled, the wire 254 extends down approximately the center of the channel in the housing 310 to a position near or extending from the distal end of the housing 310 and the ceramic tip 320. The distal face of the connector body 252 rests on shoulder 320 in housing 310 and the electrode shoulder rests on the shoulder of housing 310. The rounded or beveled portion of the connector provides a conductive surface for making a connection to connector 230. In an alternative embodiment, the active electrode may be tubular such that the inert gas can flow down the center of the tube. Such a tubular electrode also may form part or all of a telescoping assembly that allows a surgical blade or other electrode to be extended from or withdrawn from the end of the housing.

During use, an inert gas such as helium or argon flows from a gas source, through the hand piece and into the channel within the housing 310. The gas flows through the channel in the connector body 252 and down the channel in the housing 510. The gas flowing down the channel in the housing 510 surrounds the wire 254. Electrical energy is supplied from an electrosurgical generator and flows through connector 130 to wire 120, through the hand piece and various connectors to the electrode 250. As the gas flows through the attachment, the electrode connector 252 and the wire 254 highly ionize the gas so the gas becomes a cold plasma. The system is monopolar, so the attachment and hand piece include only the active electrode 250. A conductive plate (patient pad) may be placed under the patient and acts as the return electrode or ground.

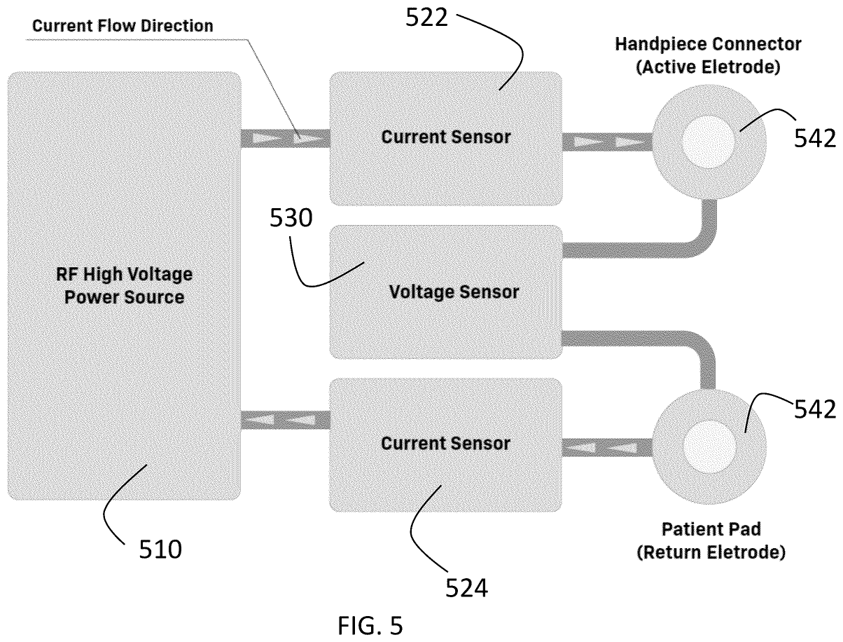

The system and method of the present invention may be used with a variety of electronic equipment used in an operating room. An exemplary gas-assisted electrosurgical generator 400 is shown in FIG. 4. The generator 400 has a power supply 402, a CPU (or processor or FPGA) 410 and a memory or storage 411. The system further has a display 520 (FIG. 5), which may be the display of a tablet computer. The CPU 410 controls the system and receives input from a user through a graphical user interface displayed on display 520. The CAP generator further has a gas control module 440 connected to a source 410 of a CAP carrier gas such as helium. The CAP generator 400 further has a radio frequency (RF) power module 450 for generating radio frequency (RF) energy. The RF power module contains conventional electronics such as are known for providing RF power in electrosurgical generators. The gas module 440 and RF power module 450 are connected to connector 460 that connects to an applicator 200.

The adaptive control system of the present invention is shown in and described with referenced to FIGS. 5-7. FIG. 5 is an overview of an adaptive non-thermal plasma control system in accordance with a preferred embodiment of the present invention. The system has a radio frequency (RF) high voltage power source 510, a current sensor 522 for sensing the current outputted by the RF high voltage source 520 to the electrosurgical handpiece (active electrode) 542 and a second current sensor 524 sensing a return current from a patient pad (return electrode) 542. The system further has a voltage sensor 530 detecting a voltage difference between the active electrode 542 and the return electrode

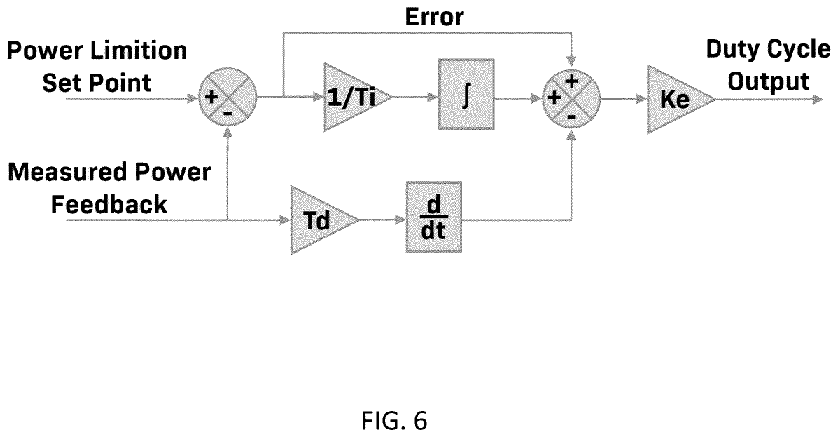

A diagram of a feedback circuit for performing the adaptive control is shown in FIG. 6. The feedback circuit preferably is implemented via an adaptive feedback control software module in +a processor or other controller in the electrosurgical generator. In a conventional electrosurgical system the current to the target tissue will increase when the active electrode is moved closer to the tissue. With the present invention, the system monitors the current to the active electrode in real time and decreases the duty cycle of the signal if the current becomes higher than the limitation set point. In this manner the plasma produced is maintained as non-thermal. The generator has a flyback transformer, which is coupled inductor with a gapped core. During each cycle, when the input voltage is applied to the primary winding, energy is stored in the gap of the core. It is then transferred to the secondary winding to provide energy to the load. The feedback circuit causes the flyback transformer to adjust the duty cycle to maintain the current in the desired range to provide non-thermal plasma.

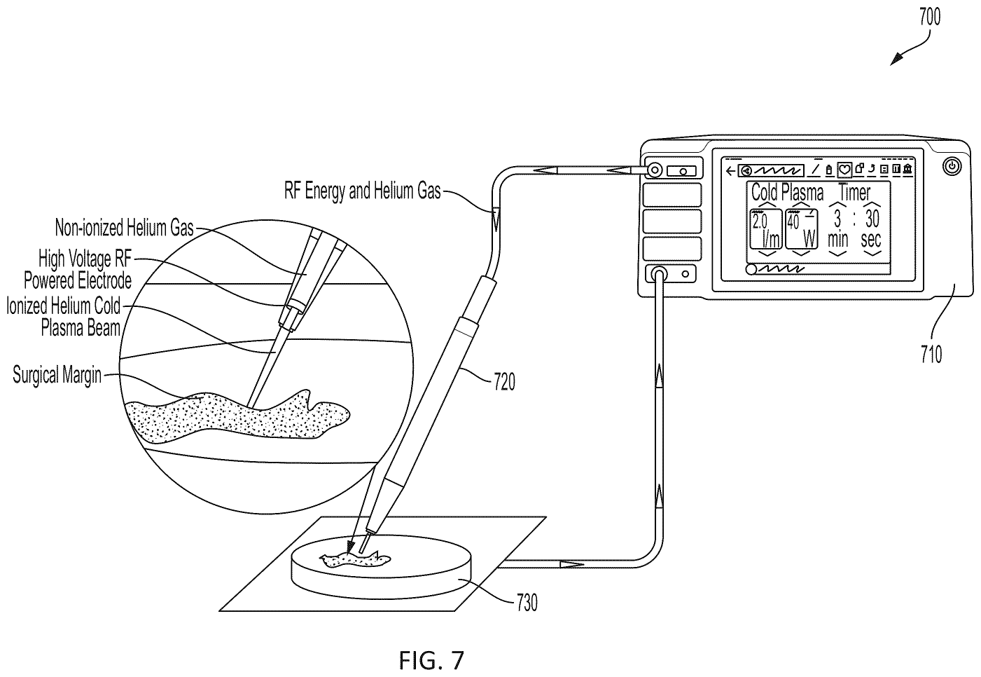

FIG. 7 is an illustration of a system having adaptive cold plasma control in accordance with a preferred embodiment of the present invention. The system 700 has an integrated electrosurgical generator 710 having a power module for supplying monopolar electrosurgical energy and a gas module for controlling flow of an inert gas such as helium. A cold plasma accessory 720 with an active electrode, such as a cold plasma scalpel, is connected to the integrated electrosurgical generator 710. A patient pad (return electrode) 730 is connected to the integrated electrosurgical generator or to a ground.

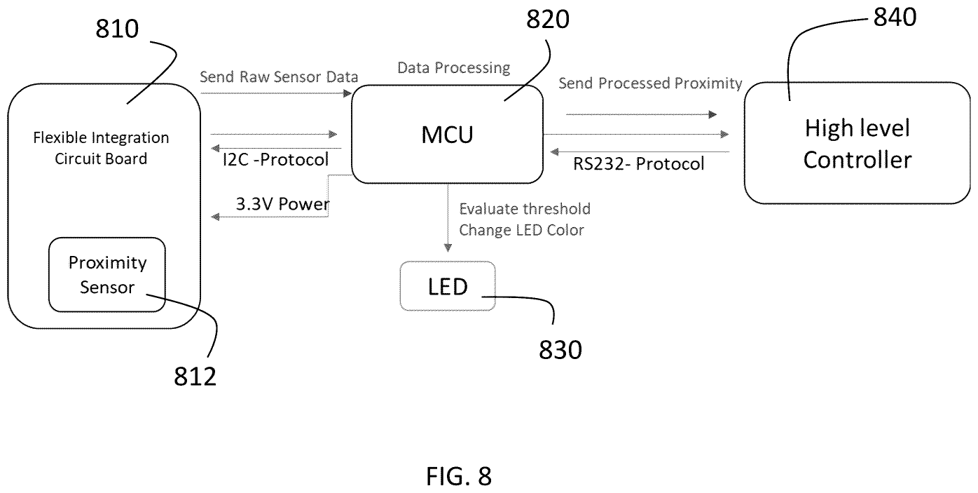

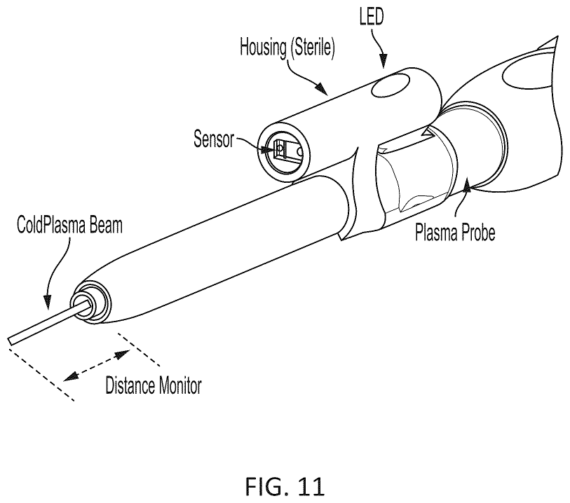

An alternate embodiment in which a proximity sensing system assisted in maintain the distance from the active electrode to the target tissue within a range to produce non-thermal, or cold, plasma is described with reference to FIGS. 8-16. While a preferred embodiment of the alternate embodiment is described herein with respect to a manual hand piece, the present invention may similarly be used with robotic gas-assisted electrosurgical systems. Similarly, while the system is described and shown in the figures in the context of an electrosurgical accessory for open surgery, the system and method equally could be used, for example, in laparoscopic or endoscopic surgery.

In the proximity sensor embodiment, a distance from the electrosurgical accessory end to the patient tissue is measured in real time. With this sensing capability, the system provides for a method to monitor if the instrument is in the optimal treatment range, i.e., a range to produce non-thermal plasma, during the surgery in real time. The system further provides cold plasma beam control, e.g., length of the plasma beam, and cold plasma treatment duration control. Still further, the system may include robotic depth mapping in 3D in the abdomen using a robotic laparoscopic device.

Different types of sensors may be used. Optical digital proximity sensors use the photodiodes to sense reflected IR energy (sourced by the integrated LED) to convert physical motion information (i.e., distance) to a digital information. Ultrasonic sensors use the principal of ultrasonic Time-of-Flight (ToF) range. Ultrasonic sensors are frequency matched Pitch-Catch products intended for applications using a first sensor for transmit and a second sensor for receiving the frequency matched ultrasonic pulse. The sensor chip my runs Chirp's advanced ultrasonic DSP algorithms and include an integrated microcontroller that provides digital range readings via I2 C. The size of these types of miniature sensors is less than 3 mm×3 mm×1 mm.

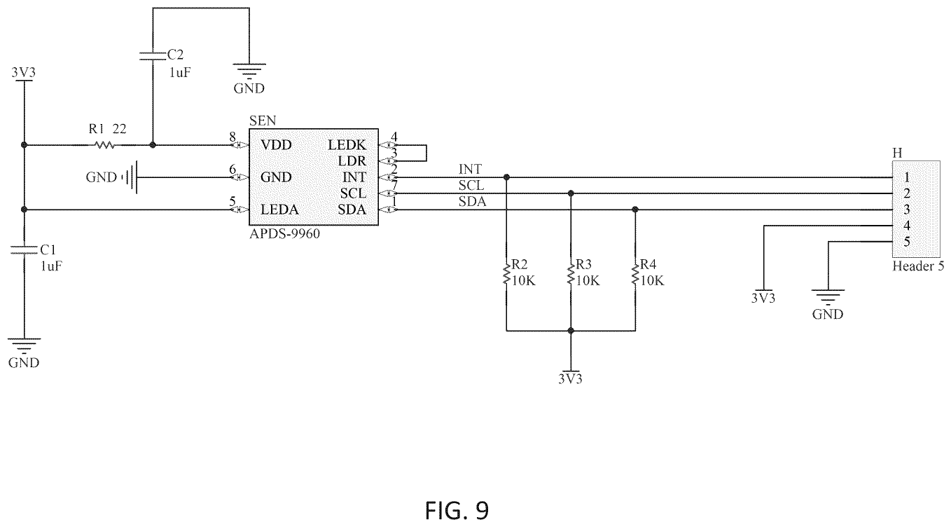

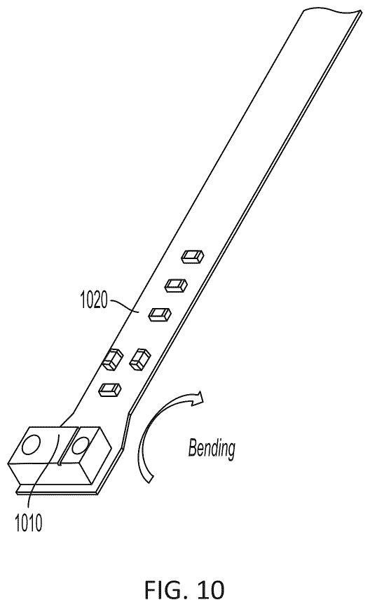

The present invention uses a new flexible sensor board shown in FIG. 10. A schematic diagram of an exemplary flexible sensor board is shown in FIG. 9. The distance sensor 1010 is mounted onto a flexible printed circuit board (PCB) 1020. The flexible PCB allows the sensor to be housed in an enclosure with very small cross-sectional area. This enables the distance measurement device to be used, for example, in laparoscopic surgical applications.

The present invention may be used in a variety of different applications.

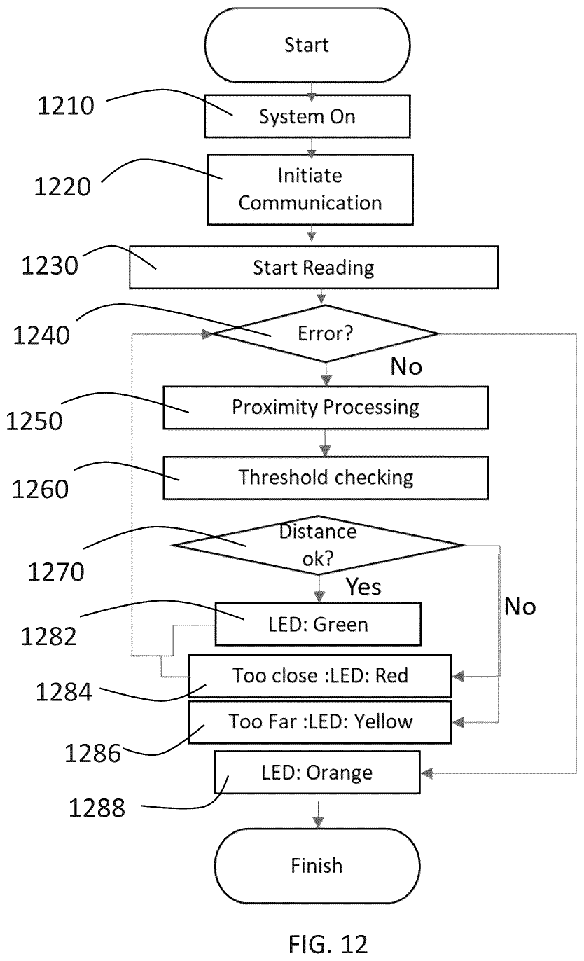

A first application is a method for a hand-held plasma treatment device as shown in FIG. 12. The system is turned on (1210), and the controller in the generator initiates communication (1220) with the flexible sensor board. The sensor starts reading (sensing) (1230) a distance from the tip of the accessory to the target tissue and determines (1240) whether there is an error in the sensor readings. If there is an error, the system gives a visible or audible signal, such as an orange LED (1288), to the user. If no error is detected, the system proceeds with proximity processing (1250) of the sensed distance and performed threshold checking (1260) to determine (1270) whether the distance is less than a first threshold or greater than a second threshold. If the distance is less that the first threshold, the system provides a visual or audible signal, for example, a red LED (1284). If the distance is greater than the second threshold, the system provides a visual or audible signal, for example, a yellow LED (1286). If the distance from the tip of the plasma accessory is within the desired range, the system may provide a visual or audible signal, such as a green LED (1282).

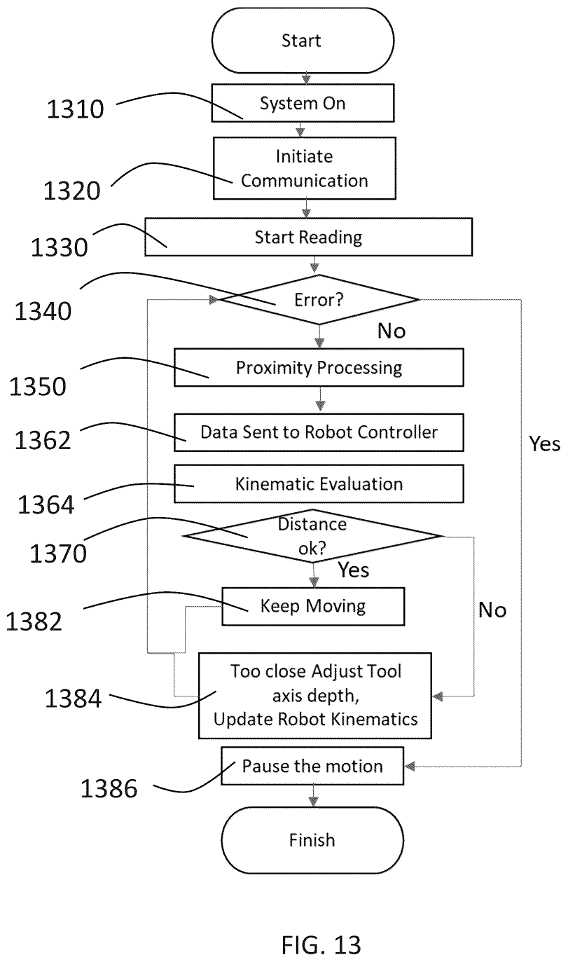

A second application is a method for robot-held plasma treatment as shown in FIG. 13. The system is turned on (1310), and the controller in the generator initiates communication (1320) with the flexible sensor board. The sensor starts reading (sensing) (1330) a distance from the tip of the accessory to the target tissue and determines 1040 whether there is an error in the sensor readings. If there is an error, the system pauses the robot and the procedure (1386), to the user. If no error is detected, the system proceeds with proximity processing (1350) of the sensed distance and the data is sent to the robot controller (1362), which performed a kinematic evaluation of the data (1364). The system then determines whether the distance is within the desired range (1370). If so, the robot continues moving on its current course (1382). If the distance is too close, the system adjusts the axis depth to move the tip of the plasma accessory further from the target tissue (and back into the desired depth range) and updates the robot kinematics (1384).

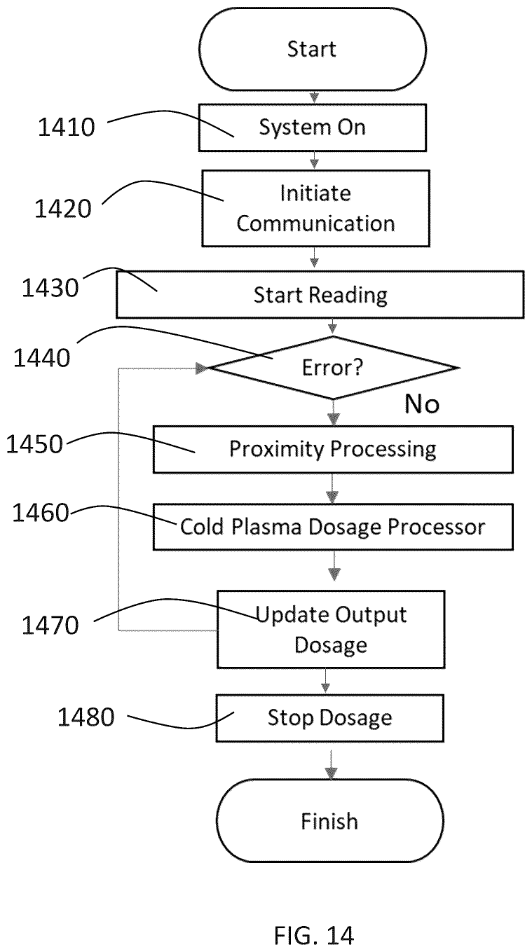

In cold plasma treatment, it is important to keep certain distance from the beam to the treatment surface to achieve the optimal clinical outcome. A third application is a method for dynamically controlling the cold plasma dosage parameters with the proximity data as shown in FIG. 14. The real-time cold plasma dosage processor dynamically finds the optimal parameters by using the real-time distance data from the tissue. The system is turned on (1410), and the controller in the generator initiates communication (1420) with the flexible sensor board. The sensor starts reading (sensing) (1430) a distance from the tip of the accessory to the target tissue and determines (1440) whether there is an error in the sensor readings. If there is an error, the system pauses the pauses the dosage. If no error is detected, the system proceeds with proximity processing (1450) of the sensed distance and the data is sent to the cold plasma dosage processor (1460). The system then updates the output dosage based on the distance data (1470). When the dosage is fully applied, the system stops the procedure (1480).

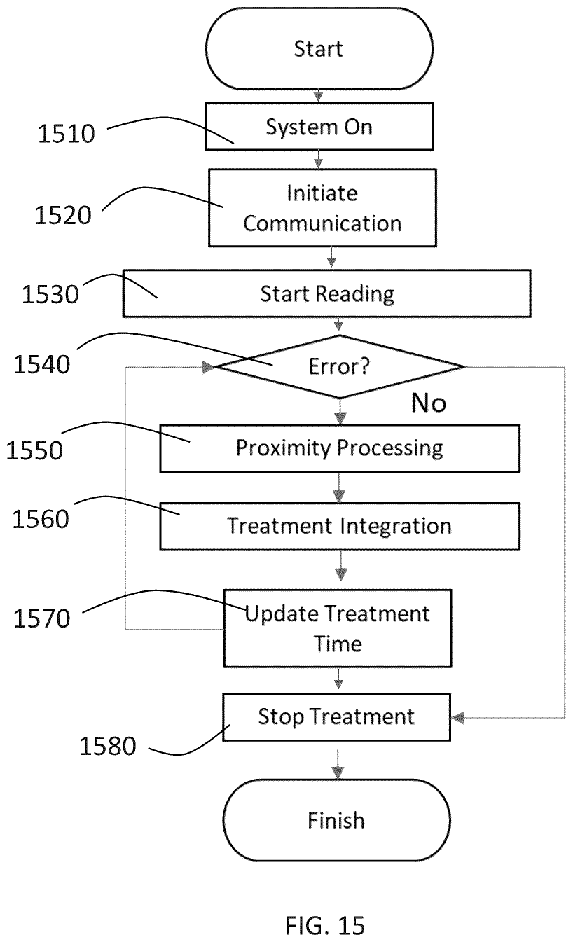

While the dosage is one of the most critical parameters for efficient cold plasma treatment, it is also important to apply the cold plasma treatment over the region for certain amount of time for best efficacy. A fourth application is a method to estimate total amount of the delivered cold plasma using treatment time, the proximity data, delivered dosage amount, and dynamic dosage parameters in shown in FIG. 15. The system is turned on (1510), and the controller in the generator initiates communication (1520) with the flexible sensor board. The sensor starts reading (sensing) (1530) a distance from the tip of the accessory to the target tissue and determines (1540) whether there is an error in the sensor readings. If there is an error, the system stops the treatment (1580). If no error is detected, the system proceeds with proximity processing (1550) and treatment integration (1560). The system then updates the treatment time based on the distance data (1570). Once the treatment time is reached, the system stops the treatment (1580).

By comparing the initially planned treatment time and the real-time treatment status, the system dynamically controls the total required treatment time.

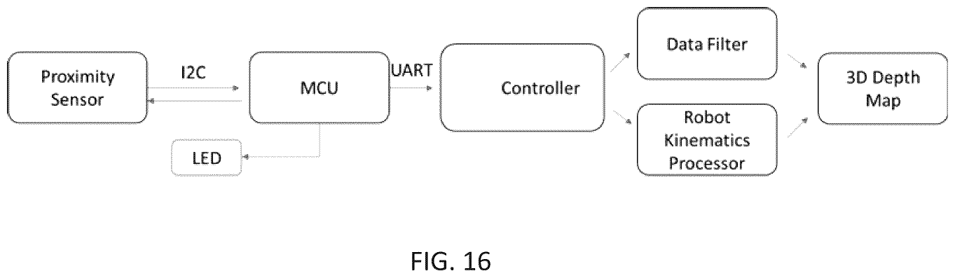

A fifth method is a robot-based surface depth map. A system for calibration of a proximity sensor equipped laparoscopic probe in accordance with a preferred embodiment of the present invention is shown in FIG. 16. To find a rigid body transformation between the sensor to the robot coordinate system, the system:
performs proximity data sampling for N positions of surgical robot;
calculates forward kinematics of surgical robot, and proximity sensor data at each position;
finds a 3D projected point on the sampling surface (depth map for each position);
repeats for all N sampling points;
develops a mesh; and
performs smoothing.

The foregoing description of the preferred embodiment of the invention has been presented for purposes of illustration and description. It is not intended to be exhaustive or to limit the invention to the precise form disclosed, and modifications and variations are possible in light of the above teachings or may be acquired from practice of the invention. The embodiment was chosen and described in order to explain the principles of the invention and its practical application to enable one skilled in the art to utilize the invention in various embodiments as are suited to the particular use contemplated. It is intended that the scope of the invention be defined by the claims appended hereto, and their equivalents. The entirety of each of the aforementioned documents is incorporated by reference herein.

1. (canceled)

2. (canceled)

3. (canceled)

4. A system for performing non-thermal plasma treatment on a patient comprising:
an electrosurgical generator for supplying radio frequency energy;
a return electrode connected to said electrosurgical generator;
a gas control module for controlling a supply of an inert gas; and
a hand piece having a proximal end and a distal end, wherein said hand piece comprises:
a housing having an inner cavity;
tubing within said cavity;
a fluid connector connecting said tubing to said gas control module;
an active electrode within said cavity;
an electrical connector connecting said active electrode to said electrosurgical generator; and
a printed circuit board within said cavity and connected to said conductor;
wherein said electrosurgical generator comprises:
a power module;
a memory;
a first current sensor configured to sense an electrical current on said active electrode;
a second current sensor configured to sense a current on said return electrode;
a voltage sensor configured to sense a voltage difference between said active electrode and said return electrode; and
a feedback control module, wherein said control module is configured to automatically adjust a power output of said electrosurgical generator based on outputs of said first current sensor, said second current sensor and said voltage sensor to maintain an electrical current on said active electrode within a range to provide non-thermal plasma;
wherein said electrosurgical generator includes a flyback transformer and said feedback control module cause the flyback transformer to adjust the duty cycle of the electrical signal to maintain the current on the active electrode in the range to provide non-thermal plasma.

5. (canceled)

6. (canceled)

7. (canceled)
